(19) United States
(12) Patent Application Publication
(10) Pub. No.: US 2026/0013868 A1
(43) Pub. Date: Jan. 15, 2026

(54) LEFT ATRIAL APPENDAGE CLOSURE DEVICES

(71) Applicant: BOSTON SCIENTIFIC SCIMED, INC., MAPLE GROVE, MN (US)

(72) Inventors: Patrick Willoughby, Shoreview, MN (US); Scott Andrew McCullough, Plymouth, MN (US)

(73) Assignee: BOSTON SCIENTIFIC SCIMED, INC., MAPLE GROVE, MN (US)

(21) Appl. No.: 19/269,705

(22) Filed: Jul. 15, 2025

Related U.S. Application Data
(60) Provisional application No. 63/671,369, filed on Jul. 15, 2024.

(51) Int. Cl. A61B 17/12; A61B 17/00

(52) U.S. Cl. CPC A61B 17/12122; A61B 17/1219; A61B 2017/00867; A61B 2017/1205

(57) ABSTRACT

A left atrial appendage closure (LAAC) device is adapted for occluding a patient's left atrial appendage (LAA). The LAAC device includes a shape memory polymer foam element that is adapted to expand from a crimped configuration for delivery to an expanded configuration after delivery and that includes, in its expanded configuration, a distal face and a proximal face. A tubular member extends proximally from the shape memory polymer foam element and defines a lumen extending through the tubular member.

### CROSS-REFERENCE TO RELATED APPLICATIONS

This application claims the benefit of priority of U.S. Provisional Application No. 63/671,369 filed Jul. 15, 2024, the entire disclosure of which is hereby incorporated by reference.

### TECHNICAL FIELD

The disclosure relates generally to medical devices and more particularly to medical devices that incorporate a shape memory foam component.

### BACKGROUND

Medical devices implanted within the heart may include left atrial appendage closure (LAAC) devices, which are intended to close off the left atrial appendage (LAA) in order to reduce the likelihood of thrombi forming in the LAA from escaping the LAA and entering the bloodstream. Thrombi that migrate through the blood vessels may eventually plug a smaller vessel downstream and thereby contribute to stroke or heart attack. Clinical studies have shown that the majority of blood clots in patients with atrial fibrillation originate in the LAA. As a treatment, medical devices have been developed which are deployed to close off the left atrial appendage. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in a left atrial appendage closure (LAAC) device that is adapted for occluding a patient's left atrial appendage (LAA). The LAAC device includes a shape memory polymer foam element that is adapted to expand from a crimped configuration for delivery to an expanded configuration after delivery, the shape memory polymer foam element including in its expanded configuration a distal face and a proximal face. A tubular member extends proximally from the shape memory polymer foam element and defines a lumen extending through the tubular member.

Alternatively or additionally, the LAAC device may further include an occlusive region.

Alternatively or additionally, the occlusive region may be disposed on the proximal face of the shape memory polymer foam element.

Alternatively or additionally, the shape memory polymer foam element may include a distal section having a first set of one or more properties and a proximal section having a second set of one or more properties that vary from those of the first set of one or more properties. The occlusive region may be disposed between the distal section and the proximal section.

Alternatively or additionally, the tubular member may extend through the shape memory polymer foam element and through the distal face such that the lumen extends through the distal face.

Alternatively or additionally, the tubular member may extend into the shape memory polymer foam element such that the lumen terminates proximal of the distal face.

Alternatively or additionally, the lumen may include a first lumen and the tubular member may further include a second lumen.

Alternatively or additionally, the first lumen may extend through the occlusive region and may extend through the distal face, and the second lumen may extend through the occlusive region and terminates proximal of the distal face.

Alternatively or additionally, the first lumen may be adapted for injecting a first material and the second lumen may be adapted for injecting a second material that is different from the first material.

Alternatively or additionally, the shape memory polymer foam element may have a first profile when in its crimped configuration and may have a second profile different from the first profile when in its expanded configuration.

Alternatively or additionally, the tubular member may be adapted to accommodate an elongate wire extending through the lumen for advancing the LAAC device through a vasculature.

Another example may be found in a left atrial appendage closure (LAAC) device that is adapted for occluding a patient's left atrial appendage (LAA). The LAAC device includes a shape memory polymer foam element that is adapted to expand from a crimped configuration for delivery to an expanded configuration after delivery. The shape memory polymer foam element includes a distal face and a proximal face when in its expanded configuration. A first elongate tubular member defines a first lumen and extends into the shape memory polymer foam element. A second elongate tubular member defines a second lumen and extends into the shape memory polymer foam element.

Alternatively or additionally, the shape memory polymer foam element may include a distal section having a first set of one or more properties and a proximal section having a second set of one or more properties that vary from those of the first set of one or more properties.

Alternatively or additionally, the first lumen may extend into the distal section and the second lumen may terminate within the proximal section.

Alternatively or additionally, the first lumen may extend through the distal face.

Alternatively or additionally, the LAAC device may further include an occlusive region that is disposed at a boundary between the distal section and the proximal section.

Alternatively or additionally, the first elongate tubular member and the second elongate tubular member include a single elongate tubular member having two or more lumens extending therethrough.

Another example may be found in a left atrial appendage closure (LAAC) device that is adapted for occluding a patient's left atrial appendage (LAA). The LAAC device includes a shape memory polymer foam element that is adapted to expand from a crimped configuration for delivery to an expanded configuration after delivery. The shape memory polymer foam element includes a proximal section having a first set of one or more properties and a distal section having a second set of one or more properties that vary from those of the first set of one or more properties. A first tubular member defines a first lumen extending into the distal section and a second tubular member defines a second lumen extending into the proximal section.

Alternatively or additionally, the LAAC device may further include an occlusive region that is disposed relative to the shape memory polymer foam element.

Alternatively or additionally, the occlusive region may separate the proximal section from the distal section.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a partial cross-sectional view of an LAA (left atrial appendage);

FIGS. 2A, 2B, 2C, 2D, 2E and 2F are partial cross-sectional views of particular profiles of various left atrial appendages;

FIG. 3 is a schematic view of an illustrative LAAC device including a shape memory polymer foam element shown in a crimped configuration;

FIG. 4 is a cross-sectional view taken along the line 4-4 of FIG. 3;

FIG. 5 is a schematic view of the illustrative LAAC device of FIG. 3, shown in an expanded configuration;

FIG. 6 is a cross-sectional view taken along the line 6-6 of FIG. 5;

FIG. 7 is a schematic view of the illustrative LAAC device of FIG. 3, showing accelerated flushing expansion;

FIG. 8 is a schematic view of using the illustrative LAAC device of FIG. 3;

FIG. 9 is a schematic cross-sectional view of an illustrative LAAC device including a shape memory polymer foam element shown in an expanded configuration with an occlusive region disposed on a proximal face of the shape memory polymer foam element;

FIG. 10 is a schematic cross-sectional view of an illustrative LAAC device including a shape memory polymer foam element shown in an expanded configuration with an occlusive region separating a distal section and a proximal section of the shape memory polymer foam element;

FIG. 11 is a schematic cross-sectional view of an illustrative LAAC device including a shape memory foam element shown in an expanded configuration and a multi-lumen tubular member;

FIGS. 12A, 12B, 12C and 12D are schematic views providing an implementation example; and

FIGS. 13A, 13B, 13C and 13D are schematic views providing an implementation example.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

A left atrial appendage closure (LAAC) device may be adapted for occluding a patient's left atrial appendage (LAA). The LAAC device includes a shape memory polymer foam element that is adapted to expand from a crimped configuration for delivery to an expanded configuration after delivery. The shape memory polymer foam element includes in its expanded configuration a distal face and a proximal face. A tubular member extends proximally from the shape memory polymer foam element and defines a lumen extending through the tubular member.

In some cases, the LAAC device may further include an occlusive region. The occlusive region may be disposed on the proximal face of the shape memory polymer foam element, for example. In some cases, the shape memory polymer foam element may include a distal section having a first set of one or more properties and a proximal section having a second set of one or more properties that vary from those of the first set of one or more properties. The occlusive region may be disposed between the distal section and the proximal section. In some cases, the shape memory polymer foam element may have a first profile when in its crimped configuration and may have a second profile different from the first profile when in its expanded configuration.

In some cases, the tubular member may extend through the shape memory polymer foam element and through the distal face such that the lumen extends through the distal face. In some cases, the tubular member may extend into the shape memory polymer foam element such that the lumen terminates proximal of the distal face. In some cases, the lumen may include a first lumen and the tubular member may further include a second lumen. In some cases, the first lumen may extend through the occlusive region and may terminate proximal of the distal face and the second lumen may extend through the occlusive region and may extend through the distal face. As an example, the first lumen may be adapted for injecting a first material and the second lumen may be adapted for injecting a second material that is different from the first material. In some cases, the tubular member may be adapted to accommodate an elongate wire extending through the lumen for advancing the LAAC device through a vasculature.

A left atrial appendage closure (LAAC) device may be adapted for occluding a patient's left atrial appendage (LAA). The LAAC device includes a shape memory polymer foam element that is adapted to expand from a crimped configuration for delivery to an expanded configuration after delivery. The shape memory polymer foam element includes, in its expanded configuration, a distal face and a proximal face. The LAAC device includes a first elongate tubular member that defines a first lumen and extends into the shape memory polymer foam element and a second elongate tubular member that defines a second lumen and extends into the shape memory polymer foam element.

In some cases, the shape memory polymer foam element may include a proximal section having a first set of one or more properties and a distal section having a second set of one or more properties that vary from those of the first set of one or more properties. In some cases, the first lumen may terminate within the proximal section and the second lumen may extend into the distal section. In some cases, the second lumen may extend through the distal face. The LAAC device may further include an occlusive region that is disposed at a boundary between the proximal section and the distal section. In some cases, the first elongate tubular member and the second elongate tubular member may together include a single elongate tubular member that has two or more lumens extending therethrough.

A left atrial appendage closure (LAAC) device may be adapted for occluding a patient's left atrial appendage (LAA). The LAAC device includes a shape memory polymer foam element that is adapted to expand from a crimped configuration for delivery to an expanded configuration after delivery. The shape memory polymer foam element includes a proximal section having a first set of one or more properties and a distal section having a second set of one or more properties that vary from those of the first set of one or more properties. A first tubular member defines a first lumen that extends into the proximal section and a second tubular member defines a second lumen that extends into the distal section. In some cases, the LAAC device may further include an occlusive region disposed relative to the shape memory polymer foam element. As an example, the occlusive region may separate the proximal section from the distal section.

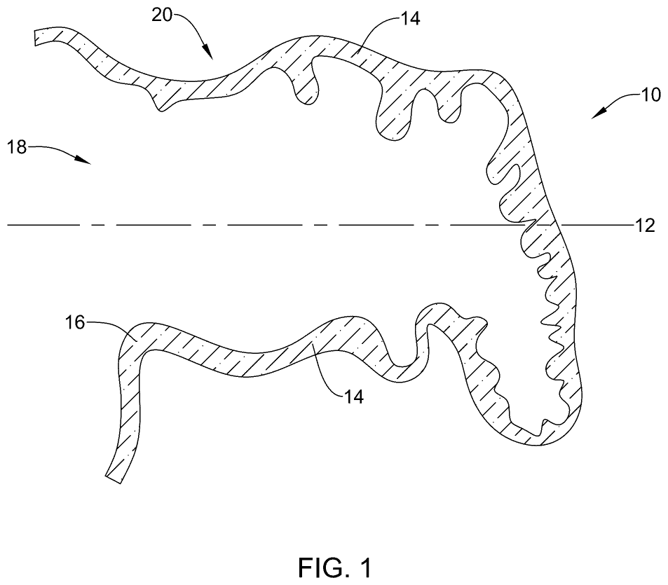

FIG. 1 is a partial cross-sectional view of a left atrial appendage 10. In some embodiments, the left atrial appendage (LAA) 10 may have a complex geometry and/or irregular surface area. It will be appreciated that the illustrated LAA 10 is merely one of many possible shapes and sizes for the LAA 10, which may vary from patient to patient. Those of skill in the art will also recognize that the medical devices, systems, and/or methods disclosed herein may be adapted for various sizes and shapes of the LAA 10, as necessary. The left atrial appendage 10 may include a generally longitudinal axis 12 arranged along a depth of a main body 20 of the left atrial appendage 10. The main body 20 may include a lateral wall 14 and an ostium 16 forming a proximal mouth 18. In some examples, a lateral extent of the ostium 16 and/or the lateral wall 14 may be smaller or less than a depth of the main body 20 along the longitudinal axis 12, or a depth of the main body 20 may be greater than a lateral extent of the ostium 16 and/or the lateral wall 14. In some examples, the LAA 10 may narrow quickly along the depth of the main body 20 or the left atrial appendage may maintain a generally constant lateral extent along a majority of the depth of the main body 20. In some examples, the LAA 10 may include a distalmost region formed or arranged as a tail-like element associated with a distal portion of the main body 20. In some examples, the distalmost region may protrude radially or laterally away from the longitudinal axis 12.

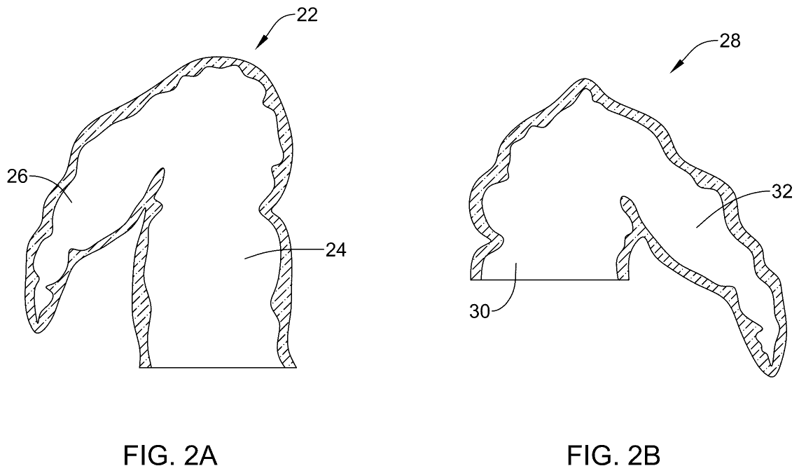

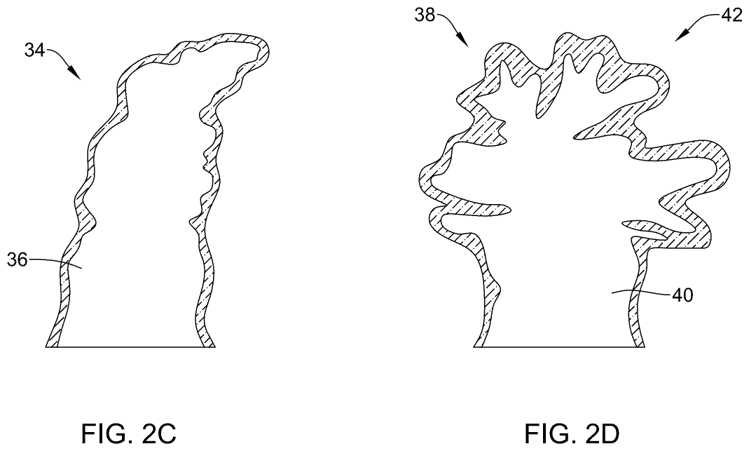

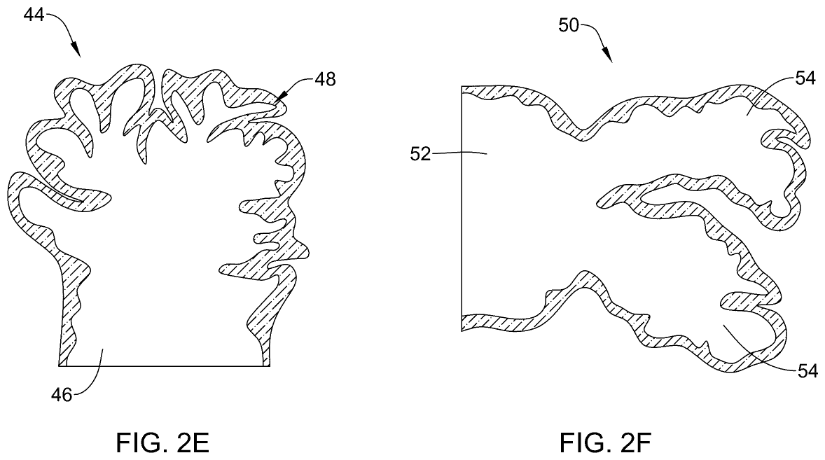

It will be appreciated that FIG. 1 shows an LAA 10 that is just one example of what a left atrial appendage may look like. While some patients may have a left atrial appendage that looks similar to the LAA 10, some patients have a left atrial appendage that may have a different shape from the LAA 10. FIGS. 2A-2F are partial cross-sectional views providing illustrative left atrial appendage shapes. It will be appreciated that this is not intended to be exhaustive, but merely illustrative, as the shape memory foam components described herein may be used in any possible left atrial appendage shape.

FIG. 2A shows an LAA 22 that is commonly referred to as having a “chicken wing” profile that includes a main segment 24 and a terminal segment 26 that may be positioned at an angle with respect to the main segment 24. FIG. 2B shows an LAA 28 that may be referred to as having a “short neck chicken wing” profile that includes a main segment 30 and a terminal segment 32 that may be positioned at an angle with respect to the main segment 30. In comparing to the LAA 22, the main segment 30 of the LAA 28 may be seen as being shorter than the main segment 24. FIG. 2C shows an LAA 34 that is commonly referred to having a “windsock” profile. The LAA 34 includes a main segment 36 that tapers distally. FIG. 2D shows an LAA 38 that is commonly referred to as having a “cactus” profile. The LAA 38 includes a main segment 40 and a number of terminal segments 42. FIG. 2E shows an LAA 44 that is commonly referred to as having a “cauliflower” profile. The LAA 44 includes a main segment 46 and a number of terminal segments 48. FIG. 2F shows an LAA 50 that is commonly referred to as having a “double lobe” profile. The LAA 50 includes a main segment 52 and a pair of terminal segments 54.

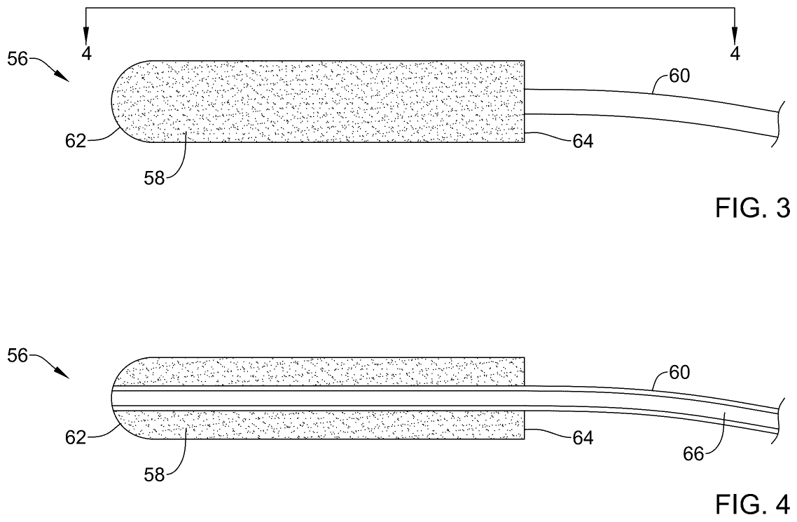

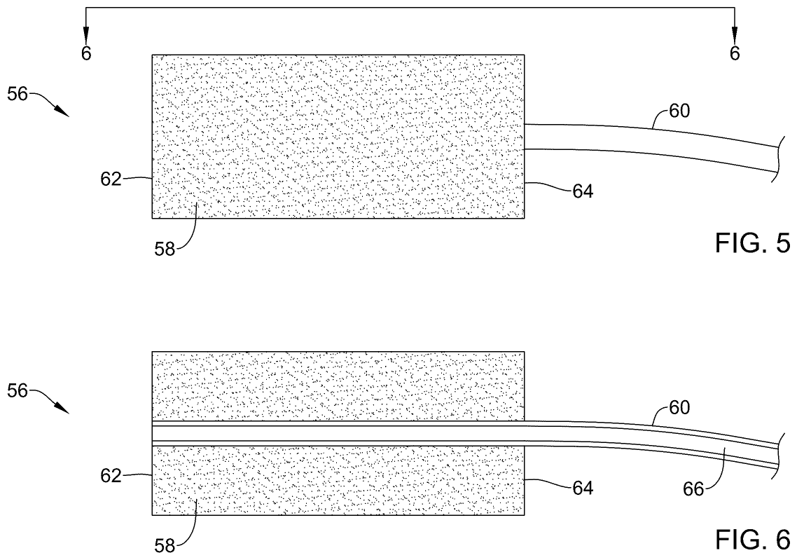

An illustrative LAAC device 56 is adapted to occlude an LAA such as the LAA 10, the LAA 22, the LAA 28, the LAA 34, the LAA 38, the LAA 44 or the LAA 50. FIG. 3 is a schematic view of the LAAC device 56 in a compressed or crimped configuration and FIG. 4 is a cross-sectional view thereof, taken along the line 4-4 of FIG. 3. FIG. 5 is a schematic view of the LAAC device 56 in an expanded configuration and FIG. 6 is a cross-sectional view thereof, taken along the line 6-6 of FIG. 5. The illustrative LAAC device 56 includes a shape memory polymer foam element 58 that is adapted to expand from a crimped configuration for delivery to an expanded configuration after delivery. The LAAC device 56 also includes a tubular member 60 that extends proximally from the shape memory polymer foam element 58. In some cases, the shape memory polymer foam element 58 may be considered as including a distal face 62 and a proximal face 64, particularly when the shape memory polymer foam element 58 is in its expanded configuration.

The tubular member 60 defines a lumen 66 that extends through the tubular member 60. In some cases, the lumen 66 may be used to accommodate a separate device such as a core wire or a guidewire that may be advanced through the lumen 66. In some cases, the tubular member 60 may be adapted to allow the LAAC device 56 to be advanced over a guidewire. As shown, the tubular member 60 (and hence the lumen 66) extends all of the way through the shape memory polymer foam element 58 such that the lumen 66 extends to the distal face 62 of the shape memory polymer foam element 58. In some cases, various materials may be injected through the lumen 66. Examples include contrast solutions, filling solutions, drug-eluting solutions, adhesives, and embolic devices. In some cases, a vacuum source may be operably coupled to the lumen 66 in order to withdraw various materials or fluids, for example. In some cases, the lumen 66 allows for cold water to be flushed through the LAAC device 56 prior to use in order to flush out any air that may otherwise exist within either the lumen 66 or within the shape memory polymer foam element 58. In some cases, the lumen 66 may accommodate advancement of tools to a position proximal of the LAAC device 56. Examples of tools include a UV curing source, an RF heater, ablation probes, pressure sensors and active electrodes. In some cases, a cutting device may be advanced through the lumen 66 that can be used for severing the tubular member 60 relative to the shape memory polymer foam element 58. In some cases, the lumen 66 may be used for injecting contrast fluid.

In some cases, the tubular member 60 may be formed from a polymeric tube. The shape memory polymer foam element 58 may be secured to the tubular member 60 in any of a variety of ways, including adhesively securing the shape memory polymer foam element 58 to the tubular member 60. The shape memory polymer foam element 58 may be crimped onto the tubular member 60, or the shape memory polymer foam element 58 may be secured to the tubular member 60 by forming the shape memory polymer foam element 58 around an end of the tubular member 60. Additional methods for securing the shape memory polymer foam element 58 to the tubular member 60 may be found in U.S. Patent Application 63/660,348, filed Jun. 14, 2024, which is herein incorporated by reference in its entirety.

In some cases, the LAAC device 56 may be advanced into a left atrial appendage such as LAA 10, the LAA 22, the LAA 28, the LAA 34, the LAA 38, the LAA 44 or the LAA 50 while the shape memory polymer foam element 58 remains in a compressed or crimped configuration. Once positioned, the shape memory polymer foam element 58 may be caused to expand into its expanded configuration. In some cases, this may involve a passive thermally driven expansion once the shape memory polymer foam element 58 warms up upon exposure to blood within and near the LAA 10, the LAA 22, the LAA 28, the LAA 34, the LAA 38, the LAA 44 or the LAA 50. In some cases, the shape memory polymer foam element 58 may have an expansion rate and a shape change that is influenced by an ability of fluid to penetrate into the shape memory polymer foam element 58 while the shape memory polymer foam element 58 is in its compressed or crimped configuration.

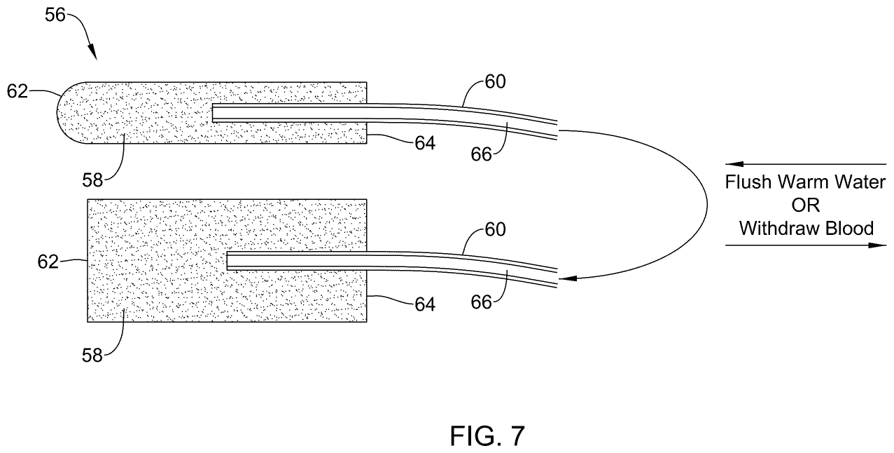

In some cases, a thermally driven expansion may be caused by actively pumping warm saline through the tubular member 60. As an example, the warm saline may have a temperature greater than body temperature (37° C.), and up to about 50° C. In some cases, a thermally driven expansion may be caused by actively pulling blood through the LAAC device 56 by applying a vacuum to the lumen 66 extending through the tubular member 60. This is illustrated for example in FIG. 7. FIG. 7 shows an example of the tubular member 60 terminating proximal of the distal face 62. This configuration may be beneficial in being able to actively cause a thermally driven expansion by either pumping warm saline into the LAAC device 56 or pulling blood through the LAAC device 56 by applying a vacuum.

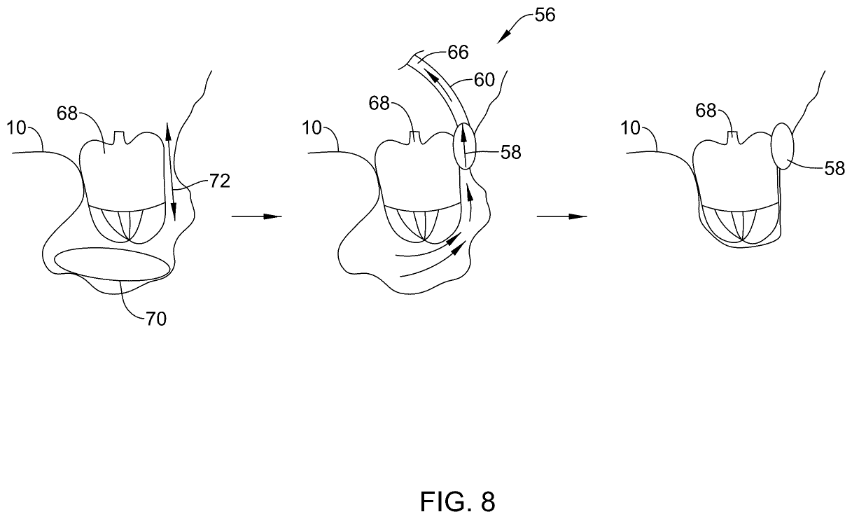

FIG. 8 provides an example of using the LAAC device 56 as a post-implant leak-filling device. On the left side of FIG. 8, another LAAC device 68 has been implanted into a left atrial appendage such as the LAA 10. Example LAAC devices 68 include WATCHMAN FLX™ and WATCHMAN FLX™ Pro available from Boston Scientific. In FIG. 8, there is a residual pool 70 of blood within the LAA 10. As shown, there is a gap 72 between the LAAC device 68 and a side wall of the LAA 10. In the middle of FIG. 8, it can be seen that the LAAC device 56 has been advanced into the gap 72, with the shape memory polymer foam element 58 disposed within the gap 72. By applying a vacuum to the lumen 66 extending within the tubular member 60, the blood originally found in the residual pool 70 is drawn towards the LAAC device 56 and is vacuumed out through the lumen 66. Once the liquid has been removed, in some cases an additional material may be injected through the lumen 66. Examples include contrast solutions, filling solutions, drug-eluting solutions, adhesives, and embolic devices. After this, the tubular member 60 may be disconnected from the shape memory polymer foam element 58, which remains behind to seal the gap 72. While FIG. 8 shows sealing a leak between the LAA 10 and the LAAC device 68, it will be appreciated that the LAAC device 56 could be used in a similar manner to seal between an aortic arch stent or graft and an aneurysm, for example.

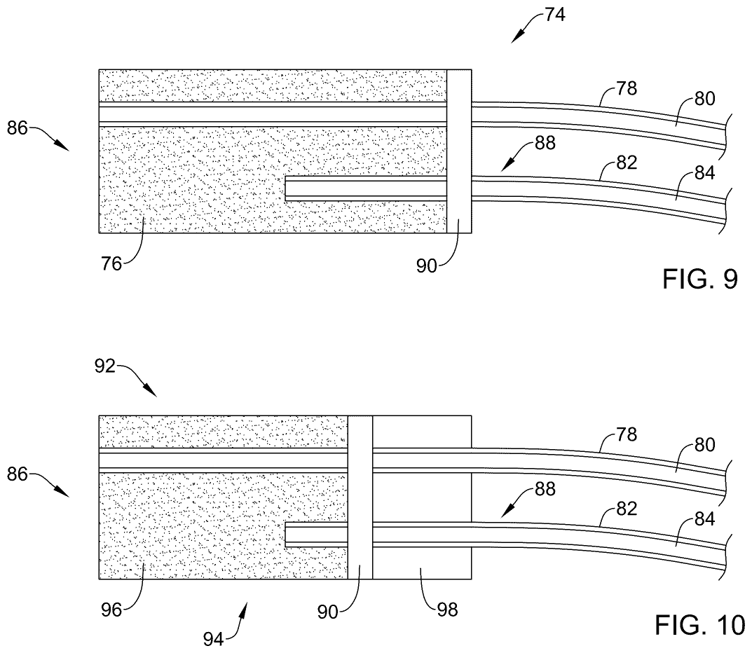

FIG. 9 is a schematic cross-sectional view of an illustrative LAAC device 74. The illustrative LAAC device 74 includes a shape memory polymer foam element 76, a first tubular member 78 defining a lumen 80 extending through the first tubular member 78, and a second tubular member 82 defining a lumen 84 extending through the second tubular member 82. As shown, the first tubular member 78 extends through the shape memory polymer foam element 76 to a distal face 86 thereof while the second tubular member 82 terminates proximal of the distal face 86. The shape memory polymer foam element 76 may be considered as including a proximal face 88. In some cases, the LAAC device 74 may include the first tubular member 78 but may not include the second tubular member 82. In some cases, the LAAC device 74 may include the second tubular member 82 but may not include the first tubular member 78.

In some cases, as shown, the LAAC device 74 may include an occlusive region 90 that is disposed on the proximal face 88 of the shape memory polymer foam element 76. The occlusive region 90 may be fully or partially occlusive. In some cases, the occlusive region 90 is more occlusive than the other foam around the occlusive region 90, such as the shape memory polymer foam element 76. In some cases, the occlusive region 90 may be a separate element that is secured to the proximal face 88. In some cases, the occlusive region 90 may represent a closed pore portion of the shape memory polymer foam element 76. The occlusive region 90 may include naturally occurring skin that is formed during the foaming process. The occlusive region 90 may include a melted added polymer or a melted or dissolved foam. The occlusive region 90 may be adhesively formed and/or adhesively secured to the proximal face 88.

FIG. 10 is a schematic cross-sectional view of an illustrative LAAC device 92. The illustrative LAAC device 92 includes a shape memory polymer foam element 94 that includes a distal section 96 and a proximal section 98. As shown, an occlusive region 90 is disposed at a boundary between the distal section 96 and the proximal section 98. Similar to FIG. 9, the LAAC device 92 includes the first tubular member 78 defining the lumen 80 extending through the first tubular member 78, and the second tubular member 82 defining the lumen 84 extending through the second tubular member 82. As shown, the first tubular member 78 extends through the shape memory polymer foam element 94 to a distal face 86 thereof while the second tubular member 82 terminates within the distal section 96. The shape memory polymer foam element 94 may be considered as including a proximal face 88. While not shown, the LAAC device 92 may include a third tubular member that terminates within the proximal section 98. In some cases, the LAAC device 92 may include the first tubular member 78 but may not include the second tubular member 82. In some cases, the LAAC device 92 may include the second tubular member 82 but may not include the first tubular member 78.

The distal section 96 and the proximal section 98 may be considered as being two different regions of foam. In some cases, the distal section 96 and the proximal section 98 may be identical in composition, apart from the relative position of the occlusive region 90. In some cases, the shape memory polymer foam within the distal section 96 may have a first set of one or more properties and the shape memory polymer foam within the proximal section 98 may have a second set of one or more properties that are different from the first set of one or more properties. The shape memory polymer foam within the distal section 96 and the proximal section 98 may differ in one or more of pore morphology, expansion characteristics, shape, size, imaging opacity and others. Depending on the chemical composition of the shape memory foam within the distal section 96 and/or the proximal section 98, which of these particular properties are most important may vary. The distal section 96 may be adapted to better accommodate the materials being injected into the foam within the distal section 96 while the proximal section 98 may be adapted to better accommodate an influx of blood into the proximal section 98.

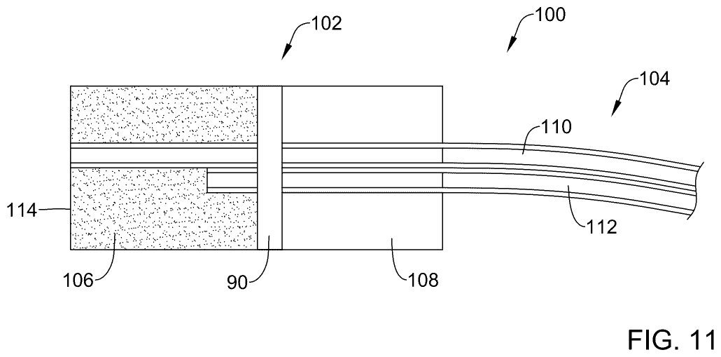

FIG. 11 is a schematic cross-sectional view of an illustrative LAAC device 100. The illustrative LAAC device 100 includes a shape memory polymer foam element 102 and a multi-lumen tubular member 104. The shape memory polymer foam element 102 includes a distal section 106 and a proximal section 108, separated by the occlusive region 90. The distal section 106 and the proximal section 108 may be considered as being two different regions of foam. In some cases, the distal section 106 and the proximal section 108 may be identical in composition, apart from the relative position of the occlusive region 90. In some cases, the shape memory polymer foam within the distal section 106 may have a first set of one or more properties and the shape memory polymer foam within the proximal section 108 may have a second set of one or more properties that are different from the first set of one or more properties. The shape memory polymer foam within the distal section 106 and the proximal section 108 may differ in one or more of pore morphology, expansion characteristics, shape, size, imaging opacity and others. As shown, the occlusive region 90 is disposed at a boundary between the distal section 96 and the proximal section 98.

The multi-lumen tubular member 104 includes, as shown, a first lumen 110 and a second lumen 112. The first lumen 110 extends through the occlusive region 90 and terminates at a distal face 114. The second lumen 112 extends through the occlusive region 90 and terminates proximal of the distal face 114, within the distal section 106. While not shown, the multi-lumen tubular member 104 may include a third lumen that terminates within the proximal section 108. Different materials may be injected using each of the first lumen 110 and the second lumen 112, and also the third lumen if included. For example, drugs or other active components could be injected into the distal section 106 using the second lumen 112 and/or injected distal of the LAAC device 100 using the first lumen 110. Coagulants may be injected to solidify blood, for example. A third lumen (not shown), if present, may be used to inject chemicals into the proximal section 108 with less risk of escape into the left side of the heart. Examples could include gels to fill the foam porosity and convert the porous structure into a solid plug, bioactive agents which could be used to solidify the blood around and through the foam to create a solid plug, adhesive materials which could be used to fix the foam plug to the inside surfaces of the LAA, and reactive foam that forms inside the remaining LAA.

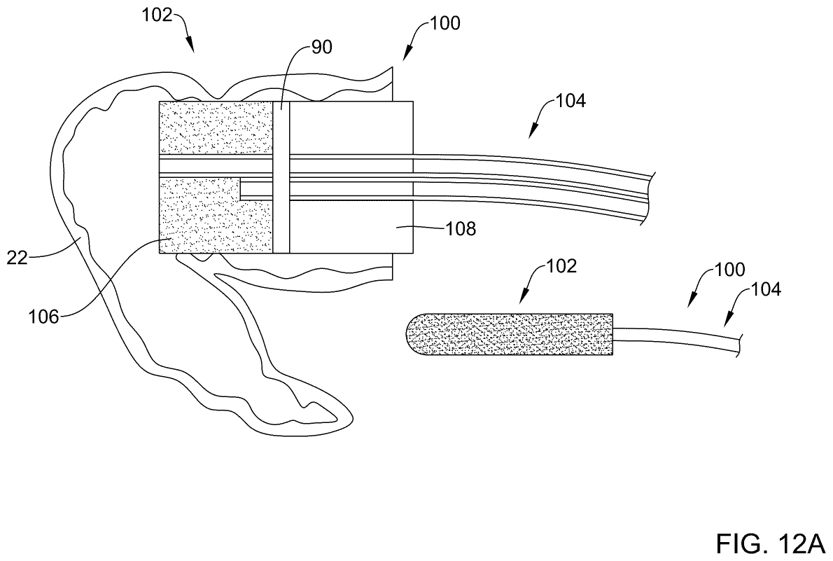

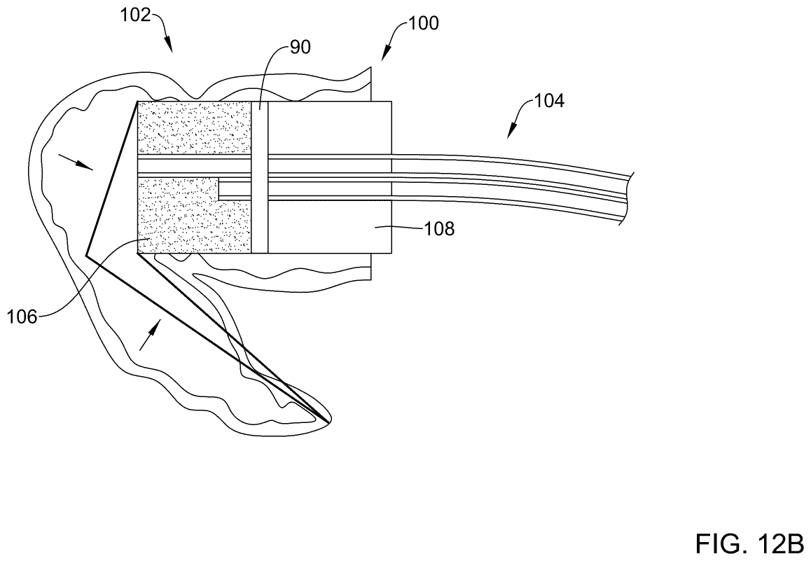

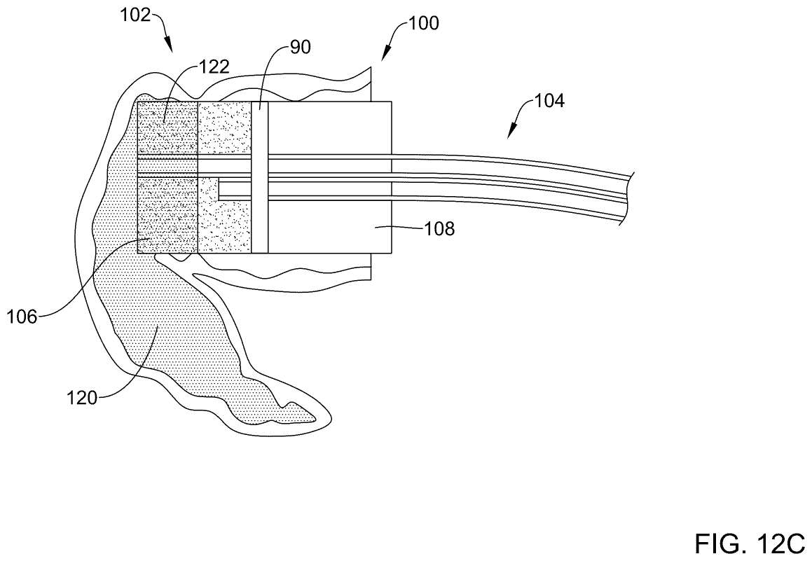

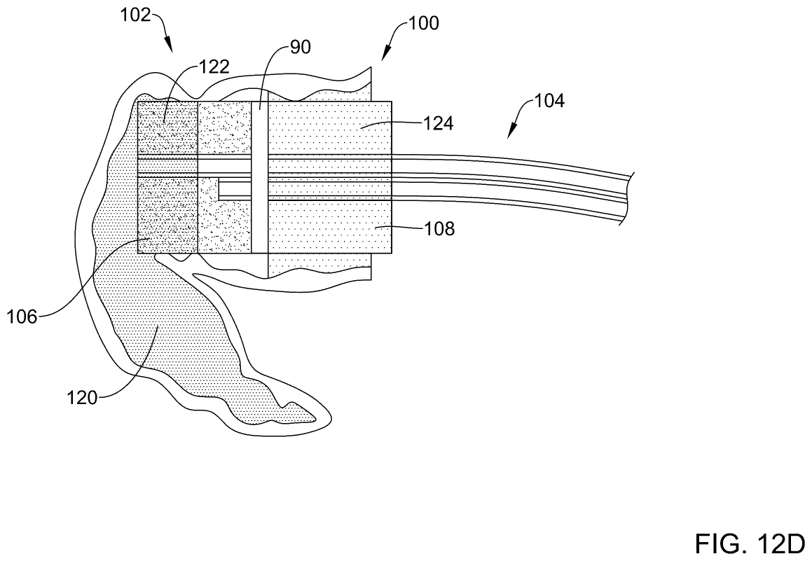

FIGS. 12A, 12B, 12C and 12D are schematic views providing an implementation example using the LAAC device 100. In FIG. 12A, the LAAC device 100 is shown in an expanded configuration after being advanced into the LAA 22 while the shape memory polymer foam element 102 was in its compressed or crimped configuration. Once positioned within the LAA 22, the shape memory polymer foam element 102 is expanded into its expanded configuration. In some cases, as shown, a source of vacuum may be coupled with the first lumen 110 in order to cause the walls of the LAA 22 to collapse inward, as shown in FIG. 12B. In some cases, this step may be skipped. Next, a fill material 120 may be injected into the LAA 22 via the first lumen 110 and the second lumen 112 may be used to inject an adhesive, a gel or a curing material 122 into the distal section 106, as shown in FIG. 12C. The fill material 120 may be a material that solidifies, adheres and/or cures in place in order to fill the volume of the LAA 22. In some cases, the proximal section 108 may be unmodified from the base foam, in order to allow for improved healing. This may be seen in FIG. 12D, where blood 124 can be seen as having infiltrated the proximal section 108. The multi-lumen tubular member 104 may subsequently be removed.

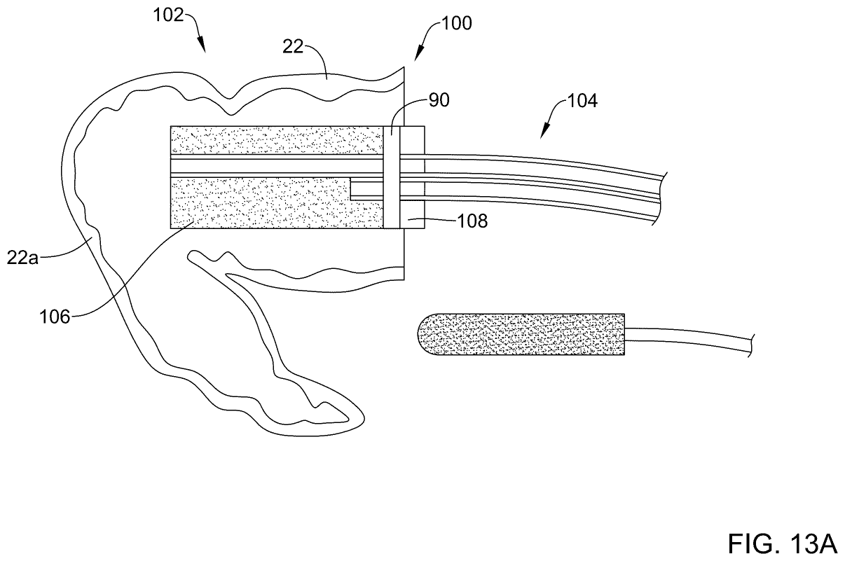

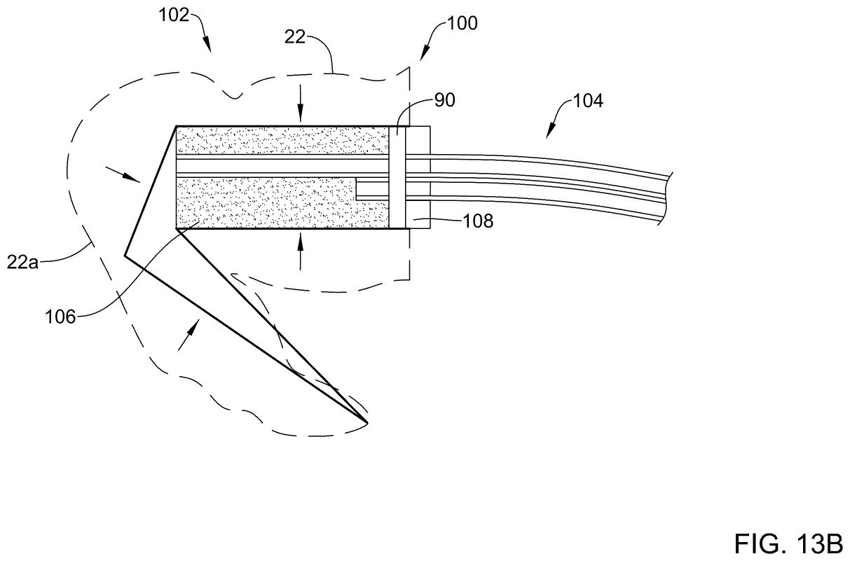

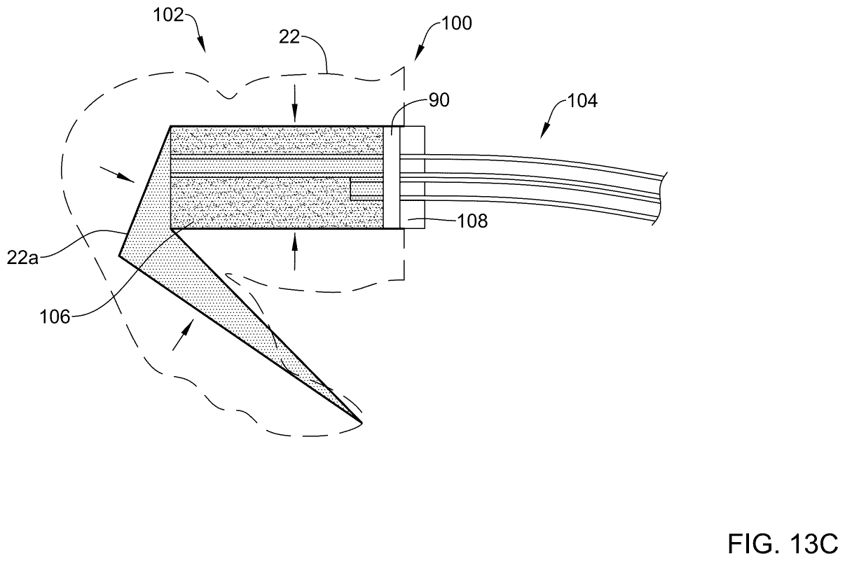

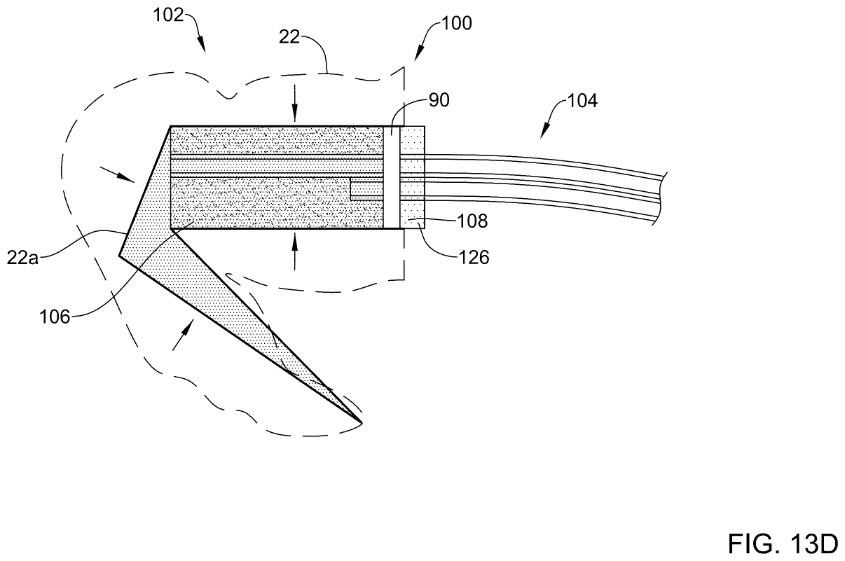

FIGS. 13A, 13B, 13C and 13D are schematic views providing an implementation example using the LAAC device 100. In FIG. 13A, the LAAC device 100 is shown in an expanded configuration after having been advanced into the LAA 22 while the shape memory polymer foam element 102 was in its compressed or crimped configuration. Once positioned within the LAA 22, the shape memory polymer foam element 102 is expanded into its expanded configuration. In some cases, as shown, a source of vacuum may be coupled with the first lumen 110 in order to pull the walls 22a of the LAA 22 to pull down onto the shape memory polymer foam element 102, as shown in FIG. 13B. In some cases, an adhesive material may be injected to adhere the walls 22a to the shape memory polymer foam element 102, as shown in FIG. 13C. In some cases, in addition to the adhesive, a radial or linear clip 126 may be added to help hold the tissue in place relative to the shape memory polymer foam element 102. In some cases, the clip 126 may be used instead of injecting the adhesive material. The clip 126 is shown in FIG. 13D. In some cases, the proximal section 108 may be unmodified from the base foam, in order to allow for improved healing. This may be seen in FIG. 12D, where blood 124 can be seen as having infiltrated the proximal section 108. The multi-lumen tubular member 104 may subsequently be removed.

The expandable foam may include any suitable material, such as a suitable polymeric material, that is capable of transitioning from an initial configuration to an expanded configuration upon being subjected to a specific temperature or temperature range and/or exposure to moisture, and provide a suitable density in the expanded configuration for use inside of the left atrial appendage to provide an occlusive benefit without negatively impacting surrounding anatomy. In some instances, the expandable foam may be a shape memory foam. Suitable transition temperatures may be, for example, at or below about 37° C. (about 98.6° F.), which allows the shape memory foam to assume an initial configuration prior to and during delivery through a delivery catheter or other delivery device, and an expanded configuration for occlusion after delivery and release within the left atrial appendage, allowing the shape memory foam to be exposed to body temperature blood within the left atrial appendage. A suitable density of the shape memory foam in the expanded configuration is a density that allows the expanded configuration to be pliable and compliant and substantially conform to the left atrial appendage anatomy to create a seal to protect against the formation and escape of blood clots while having sufficient radial force to seal the left atrial appendage but not damage or impact surrounding anatomy. In some instances, the density of the shape memory foam in the expanded configuration will be from about 10 kg/m³ (about 0.62 lb/ft³) to about 1000 kg/m³ (about 62.31 lb/ft³), including from about 10 kg/m³ to about 500 kg/m³ (about 31.2 lb/ft³) including from about 10 kg/m³ to about 200 kg/m³ (about 12.5 lb/ft³), including from about 20 kg/m³ to about 100 kg/m³ (about 6.2 lb/ft³).

Generally, the material for constructing the shape memory foam is a polymeric material that is both biocompatible and substantially biostable. In some instances, biocompatibility will include meeting or surpassing the requirements of established standards for implant materials defined in ISO 10993 and USP Class VI. Substantially biostable materials include those materials that do not resorb over the intended lifetime of the medical device (such as five years, or ten years, or longer), as well as those materials that resorb slowly such that void volume is replaced by a stable tissue-like material over a period of a few months to a year.

In some instances, the shape memory foam may include a natural and/or synthetic material. Suitable natural materials may include, for example, extracellular matrix (ECM) biopolymers such as collagen, fibronectin, hyaluronic acid and elastin, non-ECM biomaterials such as cross-linked albumin, fibrin, and inorganic bioceramics such as hydroxyapatite and tricalcium phosphate. Suitable synthetic materials may include, for example, biostable polymers such as saturated and unsaturated polyolefins including polyethylene, polyacrylics, polyacrylates, polymethacrylates, polyamides, polyimides, polyurethanes, polyureas, polyvinyl aromatics such as polystyrene, polyisobutylene copolymers and isobutylene-styrene block copolymers such as styrene-isobutylene-styrene tert-block copolymers (SIBS), polyvinylpyrolidone, polyvinyl alcohols, copolymers of vinyl monomers such as ethylene vinyl acetate (EVA), polyvinyl ethers, polyesters including polyethylene terephthalate, polyacrylamides, polyethers such as polyethylene glycol, polytetrahydrofuran and polyether sulfone, polycarbonates, silicones such as siloxane polymers, and fluoropolymers such as polyvinylidene fluoride, and mixtures and copolymers of the above.

In some instances, the shape memory foam may include a bioresorbable material such that resorption results in the formation of a biostable tissue matrix. Synthetic bioresorbable polymers may, for example, be selected from the following: (a) polyester homopolymers and copolymers such as polyglycolide (PGA; polyglycolic acid), polylactide (PLA; polylactic acid) including poly-L-lactide, poly-D-lactide and poly-D,L-lactide, poly(beta-hydroxybutyrate), polygluconate including poly-D-gluconate, poly-L-gluconate, poly-D,L-gluconate, poly(epsilon-caprolactone), poly(delta-valerolactone), poly(p-dioxanone), poly(lactide-co-glycolide) (PLGA), poly(lactide-codelta-valerolactone), poly(lactide-co-epsilon-caprolactone), poly(lactide-co-beta-malic acid), poly(beta-hydroxybutyrate-co-beta hydroxyvalerate), poly[1,3bis(p-carboxyphenoxy)propane-co-sebacic acid], and poly(sebacic acid-co-fumaric acid); (b) polycarbonate homopolymers and copolymers such as poly(trimethylene carbonate), poly(lactide-co-trimethylene carbonate) and poly(glycolide-co-trimethylene carbonate); (c) poly(ortho ester homopolymers and copolymers such as those synthesized by copolymerzation of various diketene acetals and diols; (d) polyanhydride homopolymers and copolymers such as poly(adipic anhydride), poly(suberic anhydride), poly (sebacic anhydride), poly(dodecanedioic anhydride), poly(maleic anhydride), poly[1,3-bis-(p-carboxyphenoxy)methane anhydride], and poly[alpha,omega-bis(p-carboxyphenoxy)alkane anhydride] such as poly[1,3-bis(p-carboxyphenoxy)propane anhydride] and poly[1,3-bis(p-carboxyphenoxy)hexane anhydride]; (e) polyphosphazenes such as aminated and alkoxy substituted polyphosphazenes; and (f) amino-acid-based polymers including tyrosine-based polymers such as tyrosine-based polyacrylates (e.g., copolymers of a diphenol and a diacid linked by ester bonds, with diphenols selected, for example, from ethyl, butyl, hexyl, octyl, and benzyl esters of desaminotyrosyl-tyrosine and diacids selected, for example, from succinic, glutaric, adipic, suberic, and sebacic acid), tyrosine-based polycarbonates (e.g., copolymers formed by the condensation polymerization of phosgene and a diphenol selected, for example, from ethyl, butyl, hexyl, octyl, and benzyl esters of desaminotyrosyl-tyrosine, tyrosine-based iminocarbonates, and tyrosine-, leucine- and lysine-based polyester-amides; specific examples of tyrosine-based polymers further include polymers that are comprised of a combination of desaminotyrosyl tyrosine hexyl ester, desaminotyrosyl tyrosine, and various di-acids, for example, succinic acid and adipic acid. Suitable materials include cross-linked polycarbonates and crosslinked polyethylene glycols.

In some instances, the shape memory foam may include thermoset polyurethanes that include oxidatively susceptible linkages in the soft segment, including but not limited to tertiary amines and polyethers. The shape memory foam may optionally include hydrolytically degradable soft segment components such as polycaprolactone, esters, and others. In some cases, the shape memory polymers may include non-foamed versions of the polymers described herein with respect to making the expandable foams such as shape memory foams. Example of bio-compatible shape memory polymers include polymers made from poly(F-caprolactone) (PCL), polyurethane (PU), poly (D, L-lactide) (PDLLA), PVA, ethylene vinyl acetate copolymer, (EVA) polymer blend, polymer composites, crosslinked polymers and supramolecular networks, among others. In some instances, shape memory polymers that may be used in creating the foamable solutions described herein may include polyurethane, for example.

The materials that can be used for the devices described herein may include those commonly associated with medical devices. The devices described herein, or components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, the devices described herein, or components thereof, may also be doped with, made of, or otherwise include a radiopaque material. Radiopaque materials are understood to be materials capable of producing a relatively bright image on a fluoroscopy screen or another imaging technique during a medical procedure. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of guidewire 10 to achieve the same result.

In some embodiments, a degree of Magnetic Resonance Imaging (MRI) compatibility is imparted into the devices described herein, or components thereof. For example, the devices described herein, or components thereof, may be made of a material that does not substantially distort the image and create substantial artifacts (e.g., gaps in the image). Certain ferromagnetic materials, for example, may not be suitable because they may create artifacts in an MRI image. The devices described herein, or components thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the devices described herein may be sandblasted, beadblasted, sodium bicarbonate-blasted, electropolished, etc. In these as well as in some other embodiments, a coating, for example a lubricious, a hydrophilic, a protective, or other type of coating may be applied. Alternatively, a sheath may include a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

Portions of the devices described herein may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A left atrial appendage closure (LAAC) device adapted for occluding a patient's left atrial appendage (LAA), the LAAC device comprising:
a shape memory polymer foam element adapted to expand from a crimped configuration for delivery to an expanded configuration after delivery, the shape memory polymer foam element including in its expanded configuration a distal face and a proximal face; and
a tubular member extending proximally from the shape memory polymer foam element, the tubular member defining a lumen extending through the tubular member.

2. The LAAC device of claim 1, further comprising an occlusive region.

3. The LAAC device of claim 2, wherein the occlusive region is disposed on the proximal face of the shape memory polymer foam element.

4. The LAAC device of claim 2, wherein the shape memory polymer foam element comprises a distal section having a first set of one or more properties and a proximal section having a second set of one or more properties that vary from those of the first set of one or more properties; and
wherein the occlusive region is disposed between the distal section and the proximal section.

5. The LAAC device of claim 1, wherein the tubular member extends through the shape memory polymer foam element and through the distal face such that the lumen extends through the distal face.

6. The LAAC device of claim 1, wherein the tubular member extends into the shape memory polymer foam element such that the lumen terminates proximal of the distal face.

7. The LAAC device of claim 2, wherein the lumen comprises a first lumen and the tubular member further comprises a second lumen.

8. The LAAC device of claim 7, wherein the first lumen extends through the occlusive region and extends through the distal face, and the second lumen extends through the occlusive region and terminates proximal of the distal face.

9. The LAAC device of claim 7, wherein the first lumen is adapted for injecting a first material and the second lumen is adapted for injecting a second material that is different from the first material.

10. The LAAC device of claim 1, wherein the shape memory polymer foam element has a first profile when in its crimped configuration and has a second profile different from the first profile when in its expanded configuration.

11. The LAAC device of claim 1, wherein the tubular member is adapted to accommodate an elongate wire extending through the lumen for advancing the LAAC device through a vasculature.

12. A left atrial appendage closure (LAAC) device adapted for occluding a patient's left atrial appendage (LAA), the LAAC device comprising:
a shape memory polymer foam element adapted to expand from a crimped configuration for delivery to an expanded configuration after delivery, the shape memory polymer foam element including in its expanded configuration a distal face and a proximal face; and
a first elongate tubular member defining a first lumen and extending into the shape memory polymer foam element; and
a second elongate tubular member defining a second lumen and extending into the shape memory polymer foam element.

13. The LAAC device of claim 12, wherein the shape memory polymer foam element comprises a distal section having a first set of one or more properties and a proximal section having a second set of one or more properties that vary from those of the first set of one or more properties.

14. The LAAC device of claim 13, wherein:
the first lumen extends into the distal section; and
the second lumen terminates within the proximal section.

15. The LAAC device of claim 14, wherein the first lumen extends through the distal face.

16. The LAAC device of claim 13, further comprising an occlusive region disposed at a boundary between the distal section and the proximal section.

17. The LAAC device of claim 13, wherein the first elongate tubular member and the second elongate tubular member comprise a single elongate tubular member having two or more lumens extending therethrough.

18. A left atrial appendage closure (LAAC) device adapted for occluding a patient's left atrial appendage (LAA), the LAAC device comprising:
a shape memory polymer foam element adapted to expand from a crimped configuration for delivery to an expanded configuration after delivery, the shape memory polymer foam element including a proximal section having a first set of one or more properties and a distal section having a second set of one or more properties that vary from those of the first set of one or more properties; and
a first tubular member defining a first lumen extending into the distal section; and
a second tubular member defining a second lumen extending into the proximal section.

19. The LAAC device of claim 18, further comprising an occlusive region disposed relative to the shape memory polymer foam element.

20. The LAAC device of claim 19, wherein the occlusive region separates the proximal section from the distal section.
